(19) United States
(12) Patent Application Publication
(10) Pub. No.: US 2018/0279952 A1
(43) Pub. Date: Oct. 4, 2018

(54) WIRED AUDIO HEADSET WITH PHYSIOLOGICAL MONITORING

(71) Applicant: LifeBEAM Technologies Ltd., Tel-Aviv (IL)

(72) Inventors: Zvi ORRON, Tel-Aviv (IL); Shimon HAYUN, Rosh HaAyin (IL); Jonathan APRASOFF, Kfar-Saba (IL); Omri YOFFE, Sde Warburg (IL)

(21) Appl. No.: 15/763,169

(22) PCT Filed: Oct. 6, 2016
(86) PCT No.: PCT/IL2016/051085

§ 371 Date: Mar. 26, 2018

Related U.S. Application Data
(60) Provisional application No. 62/243,954, filed on Oct. 20, 2015.

(51) Int. Cl. A61B 5/00; A61B 5/0205; A61B 5/0402; A61B 5/0476; A61B 5/0496; H04R 1/10

(52) U.S. Cl. CPC A61B 5/6817; A61B 5/02055; A61B 5/0402; A61B 5/0476; A61B 5/02427; A61B 5/721; A61B 5/7246; H04R 1/1016; A61B 5/0496

(57) ABSTRACT

A system for obtaining physiological information from a user is provided. The system includes a headset having at least one earbud which includes an audio speaker and a sensor for obtaining physiological data from a subject when positioned against a tissue region of an ear. The system further includes a microcontroller for relaying physiological data obtained from the sensor over a wired connection as a raw signal.

### FIELD AND BACKGROUND OF THE INVENTION

The present invention relates to a wired audio headset and, more particularly, to a wired audio headset which is capable of collecting and transmitting physiological data as a raw signal to a wired mobile device such as a smartphone.

As personal mobile devices such as smartphones become more ubiquitous, they are increasingly used to collect and process information about the environment and the user. Sensors such as GPS sensors, gyroscopes, barometers, accelerometers, microphones and the like enable a connected mobile device to collect useful data about the environment and user. Such data can be used to recognize commands/gestures for user inputs, provide location information and bearings for driving directions or track the user's movement for fitness and health purposes.

The new age of wearable sensors further extend the smartphone's capabilities by providing personal physiological information from wearable devices that wirelessly interface with the smartphone. Such wearable sensors can be used to monitor heart rate, blood oxygen saturation, body temperature, hydration state, blood pressure and the like. Physiological information collected from these sensors can be used by smartphone apps to a help a user achieve goals such as staying fit or healthy, being active, losing weight or self-management of chronic disease.

Wearable sensors typically include a processor for processing and transmitting the physiological information to the smartphone via a wired or wireless connection.

Complex wearable sensors having optical sensor arrays and a digital signal processor (DSP) typically include an on-board rechargeable battery for powering the processor and for transmitting the processed physiological information to the mobile device. Such information is typically transmitted over a wireless connection (e.g. BlueTooth), although heart rate sensing headsets capable of transmitting processed information over a wired audio jack connection are also known (SMS Audio BioSport earbuds).

Wearable sensors transmit data to the smartphone as a processed signal which includes the physiological information in a format suitable for Mobile applications. For example, heart rate (HR) data collected by optical sensors is processed by the processor of the wearable HR sensor to provide a heart rate signal which can be directly displayed to the user by an exercise app executed on the mobile device without further processing by the mobile device. This ensures that the HR signal can be displayed by a variety of different apps running on the smartphone.

The physiological information provided by, for example, a heart rate sensor is useful for determining the physiological state of a user, and can be correlated with other sensor data to derive additional physiological information. However, since the processed sensor signal collected by the mobile device from wearable sensors does not include raw signal data (the raw signal includes additional layers of information) a correlation at the raw signal level (between the smartphone's onboard sensors and the wearable sensor or several wearable sensors) cannot be carried out.

A raw PPG signal includes additional rheological information that is filtered out during processing. As such, raw signal correlation (referred to herein as ‘micro-correlation’) can be used to derive information that is not obtainable from processed signal correlation. Such information can be, for example, heart rate variability (HRV), respiration rate, changes in the AC level, and changes in the DC level. Such correlation can also be used during a standup test (www.azumio.com/blog/azumio/stand-up-test) to characterize the nature of the rising movement.

Micro-correlation can also be used to correlate a raw PPG signal with a raw voice signal (captured by the microphone of the mobile device) in order to detect stress or to correlate between the accelerometers of the headset and mobile device in order to analyze body movement (2 orthogonal body locations) and determine walking kinematics.

Thus there remains a need for, and it would be highly advantageous to have, a wired audio headset capable of collecting and transmitting physiological data to a mobile device as a raw digital signal thus enabling co-processing of raw signal data from various sensors in order to derive additional physiological information.

### SUMMARY OF THE INVENTION

According to one aspect of the present invention there is provided a headset comprising: (a) at least one earbud including an audio speaker and: (i) a first sensor for obtaining physiological data from the subject when positioned against a tissue region of an ear; (ii) a second sensor for obtaining movement data from the subject; (b) a microcontroller being for: (i) obtaining the physiological data and the movement data when the first sensor is positioned against the tissue region of the ear; (ii) converting a digital input signal to an analog output signal for driving the audio speaker; and (d) a digital interface for connecting the at least one earbud to a mobile device via a wire, the interface being configured for: (i) enabling the mobile device to power the microcontroller and the first sensor and the second sensor; (ii) communicating a digital audio signal from the mobile device to the microcontroller; and (iii) communicating the physiological data and the movement data converted by the microcontroller to the mobile device via the wire as a raw digital signal to thereby enable a processor of the mobile device to extract physiological and movement information from the data.

According to still further features in the described preferred embodiments the mobile device sets a sampling rate for the physiological data and the movement data buy sending a signal which is converted by the MCU to sensor command.

According to still further features in the described preferred embodiments the sampling rate is dependent on movement data or a signal to noise ratio of the physiological data and/or the movement data.

According to still further features in the described preferred embodiments the first sensor and the second sensor are positioned in or on a region of the at least one earbud subjected to a uniform movement pattern.

According to still further features in the described preferred embodiments the second sensor obtains movement data from at least two movement axes.

According to still further features in the described preferred embodiments the first sensor is an optical sensor.

According to still further features in the described preferred embodiments a light intensity, amplification and sampling rate of the optical sensor is modifiable by the mobile device through the microcontroller according to a skin tone of the subject.

According to still further features in the described preferred embodiments n optical sensor includes at least two light emitting diodes (LEDs).

According to still further features in the described preferred embodiments each of the at least two LEDs is capable of emitting light at a different wavelength.

According to still further features in the described preferred embodiments each of the at least two LEDs is positioned at a different distance from a photodetector.

According to still further features in the described preferred embodiments positioning the first sensor in an ear of the subject generates a signal for executing an action in the mobile device.

According to still further features in the described preferred embodiments the physiological data includes heart rate data.

According to still further features in the described preferred embodiments the physiological data includes SpO₂ data, blood pressure data, heart rate data, body temperature data and/or bioimpedance data.

According to still further features in the described preferred embodiments the first sensor is selected from the group consisting of a temperature sensor, a galvanic skin resistance sensor, a blood pressure sensor, an ECG sensor, an EOG sensor, an EEG sensor and a bioimpedance sensor.

According to still further features in the described preferred embodiments the second sensor is selected from the group consisting of an accelerometer, a gyroscope, magnometer.

According to another aspect of the present invention there is provided a system comprising: (a) a headset having at least one earbud including: (i) an audio speaker; (ii) a sensor for obtaining physiological data from a subject when positioned against a tissue region of an ear; (iii) a microcontroller for obtaining the physiological data when the sensor is positioned against the tissue region of the ear and relaying the physiological data over a wired connection as a raw signal; and (b) a mobile device having an on-board sensor and being wired to the headset via a digital interface, wherein a processor of the mobile device processes the raw signal along with a second raw signal from the on-board sensor to thereby derive information resulting from a correlation between the raw signal and the second raw signal.

According to still further features in the described preferred embodiments mobile device powers down the microcontroller and the first sensor when physiological data is not obtained.

According to still further features in the described preferred embodiments headset further comprises a movement sensor.

According to still further features in the described preferred embodiments mobile device powers up the microcontroller to query the first sensor when movement data is obtained by the movement sensor and/or when the physiological data is obtained by the first sensor.

According to still further features in the described preferred embodiments mobile device sets a sampling rate for the physiological data and the movement data.

According to still further features in the described preferred embodiments sampling rate is dependent on movement data or a signal to noise ratio of the physiological data and/or the movement data.

According to still further features in the described preferred embodiments physiological sensor is an optical sensor.

According to still further features in the described preferred embodiments light intensity, amplification and sampling rate of the optical sensor is modifiable by the mobile device according to a skin tone of the subject.

According to still further features in the described preferred embodiments the microcontroller is further configured for converting signals from one sensor or the first sensor and the second sensor to a digital format of the digital interface.

The present invention successfully addresses the shortcomings of the presently known configurations by providing a headset capable of collecting physiological data from a user and transmitting the data to a mobile device as a raw signal over a wired connection.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE SEVERAL VIEWS OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates a headset having two earbuds and a digital power interface for connecting the headset to a mobile device.

FIG. 2a is a cutaway view of one embodiment of the headset of the present invention showing the internal components of the physiological monitoring system.

FIG. 2b illustrates a typical USB interface which can be used to connect the present headset to a mobile device.

FIG. 2c is a cutaway view of another embodiment of the headset of the present invention showing the internal components of the physiological monitoring system and positioning of the MCU in a controller.

FIG. 3 is a block diagram of the physiological monitoring system of the present headset and a mobile device.

FIG. 4 is block diagram of an embodiment of the present headset in which the physiological monitoring system is connected to a single port of a mobile device.

FIG. 5 is block diagram of an embodiment of the present headset in which the physiological monitoring system is connected to two ports of a mobile device.

FIGS. 6a-c are flowcharts illustrating the processes of signal calibration and collection by the mobile device connected to the present headset.

FIG. 7 illustrates a smartphone which can be connected to the present headset via a wired digital connector (e.g. USB wire).

### DESCRIPTION OF THE PREFERRED EMBODIMENTS

The present invention is of a headset which can be used to monitor one or more physiological parameters of a user. Specifically, the present invention can communicate a raw optical PPG signal to a connected mobile device thus enabling the mobile device to extract heart rate as well as other physiological parameters from the signal.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Prior art earbuds having PPG sensors include an on-board digital signal processor (DSP) for processing raw signal data obtained by the sensors and providing a processed physiological signal to a mobile device. Since the processed signal is mathematically manipulated and filtered for a specific physiological parameter (e.g. HR), most of the information present in the raw signal obtained by the sensors is discarded during processing and not communicated to the mobile device.

Studies have shown that raw signal data obtained from multiple sensors can be co-processed and correlated to derive information which can otherwise not be obtained from single sensor data. For example, correlation of raw data from multiple sensors can be used to more accurately identify and acquire military targets (www.au.af.mil/au/awc/awcgate/vistas/sench3.pdf).

The present inventors identified a similar need in physiological monitoring systems and have devised a headset for physiological monitoring that, unlike prior art headsets, can be used in raw signal correlation applications.

Thus, according to one aspect of the present invention there is provided a headset for collecting and communicating raw signal data to a mobile device.

As used herein, the term “headset” refers to any head/neck worn gear that includes one or more earbuds (also referred to as earpieces or earplugs) which are positionable in an ear of a subject (user).

The headset of the present invention includes at least one earbud having an audio speaker. The audio speaker can be an in-ear headphone speaker such as A002A CE-058NTG 14Ω (CHIA PING ENTERPRISE CO, LTD.) (size—5.8 by 4.3 mm) and is preferably enclosed within a housing of the earbud, but can also be positioned external to the housing. The at least one earbud (also referred to herein as “first earbud”) also includes a first sensor (also referred to herein as physiological parameter sensor) for obtaining physiological data from a subject when the first earbud is positioned in an ear of the subject and the sensor is positioned against a tissue region of an ear. Such a sensor can be a PPG sensor which is positionable against a tissue region such as the tragus or any other sensor capable of obtaining HR data, SpO₂ data, blood pressure data, body temperature data and/or bioimpedance data. A PPG sensor can include one or more light emitting diodes (LEDs) and one or more photodetectors positioned at an equal or different distance from one or more LEDs. The LEDs can transmit at the same wavelength or a different wavelength within a range between UV visible and IR. Examples of PPG sensors that can be used with the present invention include, but are not limited to ADPD142 (by Analog Devices) or Max86160 (by Maxim Integrated) or the sensor array described in US20150065889.

The first earbud can also optionally include a second sensor for obtaining movement data from the subject, this sensor is positioned within the housing but can alternatively be positioned on the housing. The second sensor can be an accelerometer, a gyroscope or a magnometer. The second sensor is preferably positioned at a region of the housing that is subjected to the same movement of the physiological sensor. For example, the physiological and movement sensors can be mounted on the same subassembly of the housing or be attached no more than 20 mm apart at a region experiencing the same movement.

Additional sensors such as a temperature sensor, a galvanic skin resistance sensor, a blood pressure sensor, an ECG sensor, an EOG sensor, an EEG sensor and a bioimpedance sensor can also be included in or on the housing of the first earbud. In the case of a dual earbud headset, some sensors can be positioned in or on the housing of the second earbud. Thus, for example, the PPG and movement sensors can be positioned in/on the first earbud, while sensors such as the temperature sensor, the galvanic skin resistance sensor, the blood pressure sensor, the ECG sensor, the EOG sensor, the EEG sensor and/or the bioimpedance sensor can be positioned in/on the second earbud.

The first earbud also includes a microcontroller (MCU) positioned within the housing and connected to the first and optionally second sensors. An alternative configuration of the present headset can include the MCU in a control unit positioned on the headset cable.

The MCU is an ASIC that allows data transfer between the sensors and the mobile device and streaming of audio signals from and to the mobile device including converting audio between digital and analog formats. Thus, the MCU of the present headset is a bridge between different communication buses (e.g. case I2C to USB, USB to Audio).

The MCU of the present headset performs two main functions: (i) conversion of a digital audio signal from the mobile device to an analog signal for the speaker(s) and optionally conversion of an analog microphone signal to a digital signal for the mobile device (functions similar to those provided by Realtek ALC4040 or C-media CM6510B) (ii) conversion of data (signal) from the mobile device for configuring and operating the sensors from a first format (mobile device) to a second format (sensor) and vice versa for raw data acquisition from the sensors. For example, data from the mobile device to the sensors can be provided to the MCU as write and read commands for each sensor. Each sensor has a predefined address (i.e. I2C_Add) and in each sensor a set of registers are configured such that each register has a predefined address (RegAdd). Exemplary mobile device commands which can be used to configure the sensor registers include, but are not limited to:
OpticSensor_WriteRegister (u8 I2C_Add, u8 RegAdd, u8 Data),
OpticSensor_ReadRegister (u8 I2CAdd, u8 RegAdd, *u8 Data),
MotionSensor_WriteRegister (u8 I2CAdd, u8 RegAdd, u8 Data) and
MotionSensor_ReadRegister (u8 I2C_Add, u8 RegAdd, *u8 Data).

Raw data from sensors can be provided to the mobile device by the MCU as read commands from the sensors' data address. Exemplary mobile device commands which can be used to retrieve data from the MCU include, but are not limited to:
MotionSensor_ReadRawData (u8 I2C_Add, u8 RegAdd, *u8 Data[0], U8 Length) and
OpticSensor_ReadRawData (u8 I2C_Add, u8 RegAdd, *u8 Data[0], U8 Length).

The MCU of the present headset is configured for: (i) receiving a signal (physiological/movement sensor data) from the first sensor and optionally the second sensor when the first sensor is positioned against the tissue region of the ear and converting the signal from a first data bus to a second data bus (ii) converting a digital input signal form a connected mobile device to an analog output signal for driving the audio speaker of the earbud(s).

The MCU and sensors of the present headset are also referred to herein collectively as “physiological monitoring system”.

The headset of the present invention also includes a digital interface (connector) for connecting the first and second earbuds to a mobile device via a wire. Such a digital interface is configured for providing digital communication to and from the mobile device and for providing power from the mobile device. Examples of a digital interface for wired connection include universal serial bus (USB), Apple's Lighting™ interface, or any other proprietary connector which carries a digital signal.

In order to power the physiological monitoring system of the present headset, and provide the aforementioned data transmission between the MCU and sensors and the MCU and mobile device, the digital power connection provides at least 1.8V (1.8-4.7V typical) to the headset. Data connectivity between the MCU and mobile device can be through a USB bus at 1Hz-1KHz while the MCU and sensors can communicate through a I2C, UART or I2S communication buses.

The digital interface of the present headset is configured for: (i) enabling the mobile device to power the microcontroller and the first and second sensors; (ii) communicating a digital audio signal from the mobile device to the microcontroller and communicating a microphone signal from the MCU to the mobile device; and (iii) communicating the physiological data and movement data obtained by the microcontroller to the mobile device via a wired connection as a raw digital signal suitable for the mobile device.

The mobile device can have any number of ports (e.g., 3, 4, 5, etc.) and/or a variety of types of port (e.g., dedicated power port, dedicated data port, etc.). The present headset can be connected to at least one port of the mobile device that provides bidirectional data and power. The headset can alternatively include two interfaces, one connectable to a data port of the mobile device and one connectable to an audio port of the mobile device.

The mobile device, which can be a smartphone such as an iPhone™, or a Samsung Galaxy™, includes a processor (main processor or a dedicated DSP) for processing the raw signal(s) and extracting parameter data therefrom. The processor of the mobile device can also correlate between raw signals obtained from the various headset sensors and well as correlate/fuse raw data obtained from the first sensor and sensors of the mobile device (e.g. accelerometer/gyroscope of the mobile device).

Thus, an object of the present invention is to provide a portable physiological monitoring headset that can provide physiological data as a raw signal to a connected mobile device. The headset can send the raw physiological signal (and/or other signals) over a wired connection to a mobile device (e.g. smartphone), which may then calculate physiological parameters (e.g., heart rate, heart rate variability, temperature, etc.) and display the parameters to a user or correlate various raw signals to derive additional or more accurate information. The headset can include additional functionality (e.g., music from speakers, telephone calls via microphone and speaker, etc.) from the mobile device over the wired connection. Thus, the present headset provides dual functionality, audio and physiological monitoring. Since the present headset eliminates the need for an on-board DSP and/or power source, it also provides the benefits of decreased cost, size, and complexity.

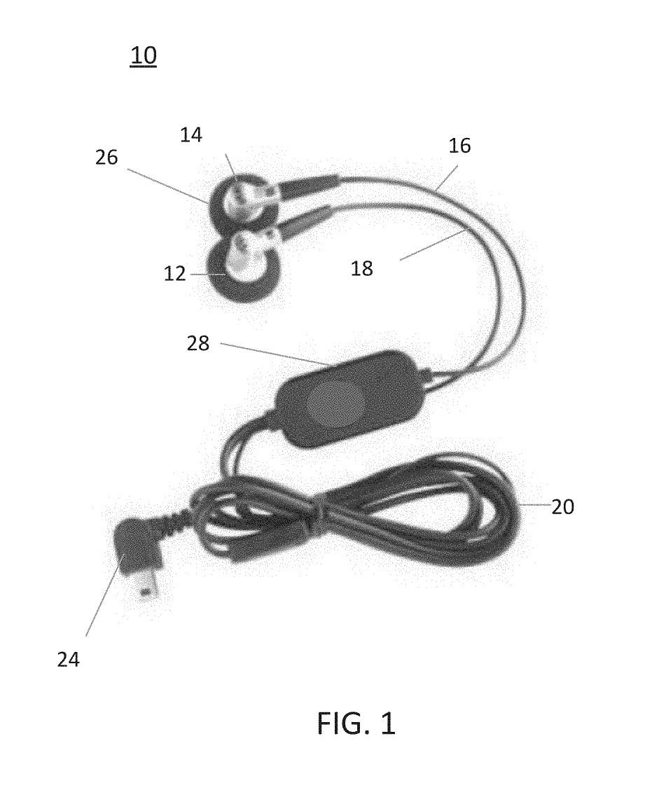

Referring now to the drawings, FIG. 1 illustrates a headset 10 which includes a physiological monitoring system and a digital connector for connecting headset 10 to a mobile device such as a smartphone.

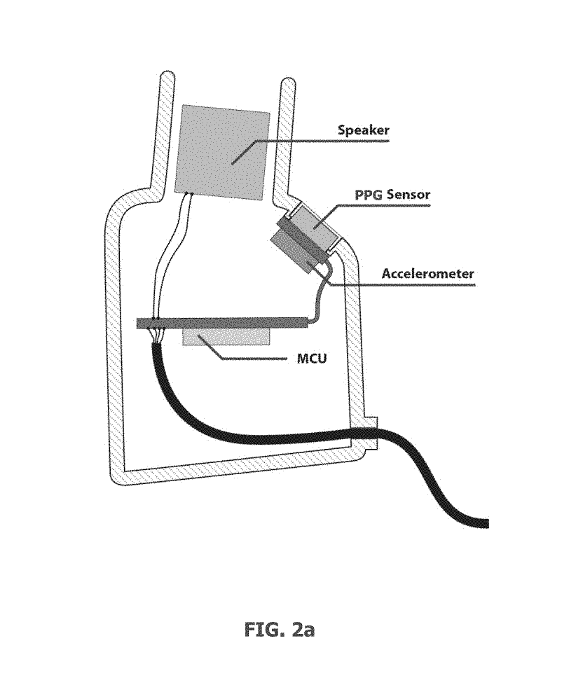

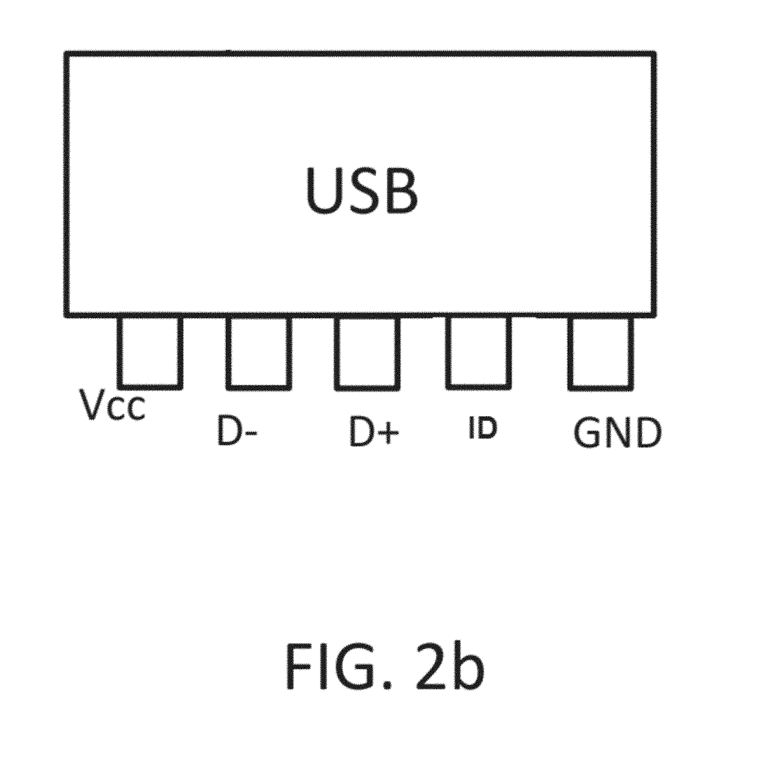

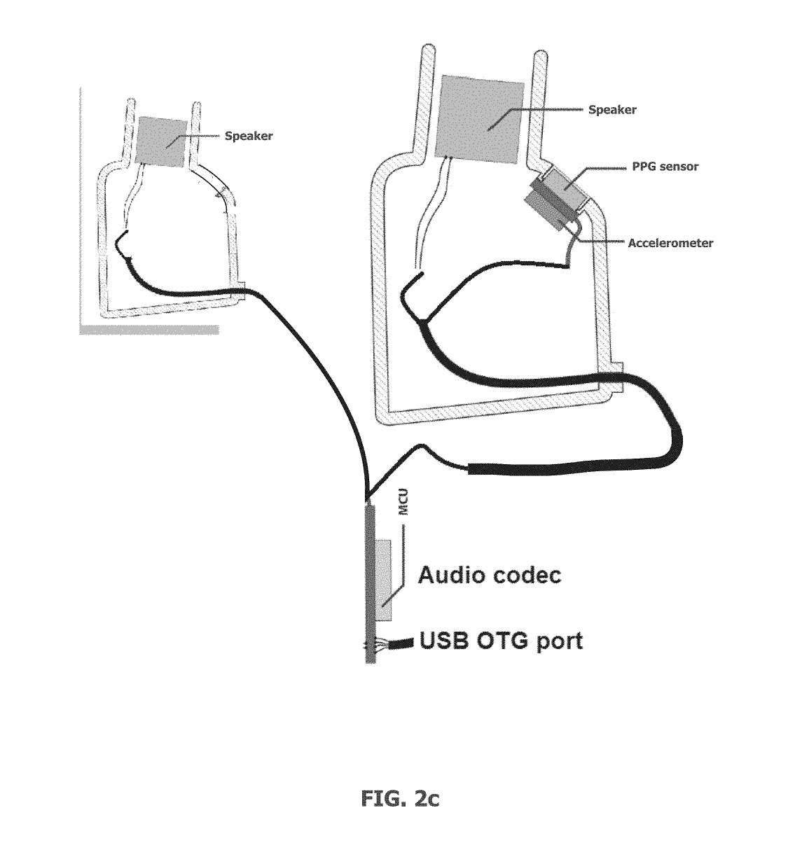

Headset 10 includes a first earbud 12, a second earbud 14, one or more cables 16, 18, and 20, collectively wire 21, and a plug 24. First earbud 12 and/or second earbud 14 include a housing 26 for enclosing one or more speakers, one or more physiological sensors (not shown), a digital power interface (for connecting to cable 16 or 18) and the MCU (FIG. 2a). Housing 26 can also enclose a power regulator. The MCU and digital power interface can alternatively be housed in control unit 28 (FIG. 2c) which includes audio controls, a microphone and the like.

Plug 24 can be any connector capable of interfacing with a desired data and power input/output port of a mobile device. For example, plug 24 can be a micro USB connector, mini-USB, a USB On the Go connector, an Apple Lighting® connector, or any input/output connector capable bidirectional data transmission and receiving input power from the mobile device.

FIG. 2a illustrates the internal components within housing 26 of earbud 12 or 14. In this embodiment of headset 10, MCU 62 is housed within the earbud and is wired to an accelerometer, a physiological sensor (e.g. PPG) and the speaker. The MCU is also connected to the digital power interface which is in turn connected via cable and plug to the digital power port of the mobile device.

FIG. 2c illustrates a configuration in which the MCU (with integrated digital to analog audio codec) resides within a control unit (volume, on/off control etc) positioned on the cable connecting the headset to a mobile device (e.g. USB cable).

FIG. 2b is block diagram of an exemplary USB on the go (OTG) connector. The OTG connector includes a power pin (Vcc), two data pins D− and D+, an ID pin and ground (GND). In some embodiments of the present headset, the circuitry associated with the physiological monitoring system is connected to the Vcc pin for power, and data is transmitted bi-directionally between the mobile device, and the speakers and physiological sensor using data pins D− and D+. The ID pin is connected to GND to indicate to the mobile device to switch to HOST mode, otherwise (if PIN ID left floated) the mobile device will stay in client mode. It will be appreciated that other configurations of the power and data pins can also be used. Embodiments using solely a digital data connection may require a digital to analog converter in order to convert digital audio data from the smartphone to an analog audio signal for the speakers in the earbuds. Such digital to analog conversion can be handled by the MCU or a dedicated digital to analog converter.

As is mentioned hereinabove, embodiments of headset 10 can include two connectors, a first connector to transmit digital data and receive power when connected to a data/power input/output port of a mobile device, and a second connector to receive analog audio signals from an audio port (3.5 audio connector) of a smartphone. Such embodiments of headset 10 would not require a digital to analog converter since audio signals transmitted from the mobile device are in an analog format.

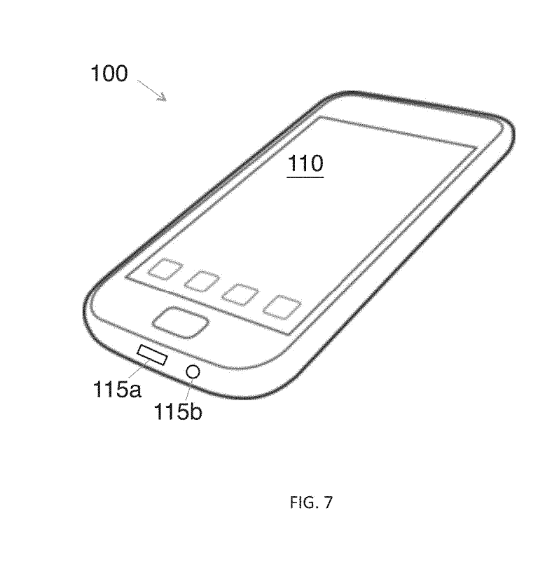

To use headset 10 the plug 24 is inserted into an input/output port of a mobile device (e.g., input/output 115a of smartphone 100 shown in FIG. 7) to provide power and data connection to headset 10. Earbuds 12 and 14 are then placed into an ear of a user. Positioning of earbud 12 or 14 in the ear of the user activates the MCU (via PPG sensor which detects skin contact or a dedicated optical skin contact sensor) to collect data from the various physiological sensors within earbuds 12 and/or 14. The physiological signal measurements are transmitted to smartphone 100 via data pins in the plug 24 and the input/output port 115b of the smartphone 100. Audio data (e.g., music, phone calls) may be transmitted to the smartphone 100 via data pins in the plug 24 and the input/output port 115b of the smartphone 100.

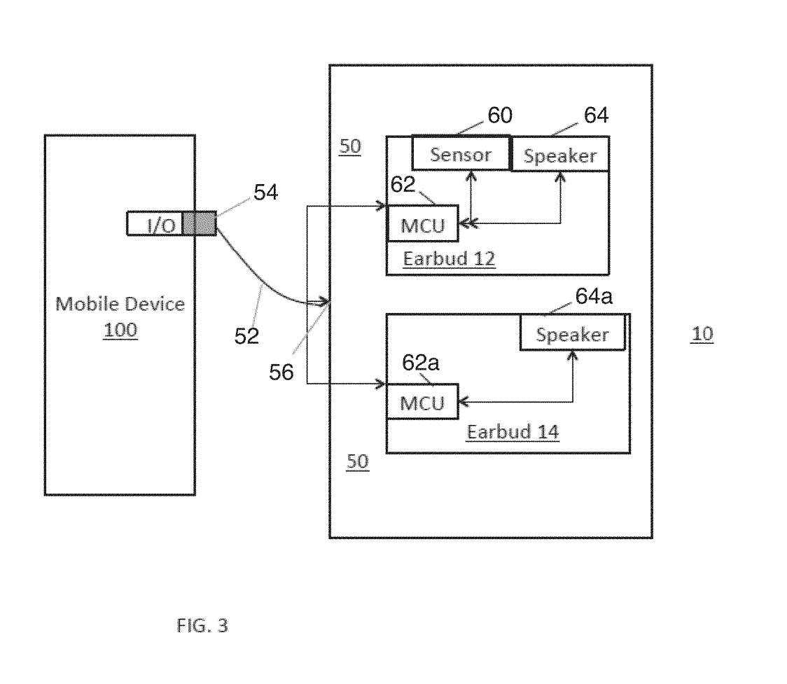

FIG. 3 is block diagram of a two earbud headset 10 with a single earbud including the physiological monitoring system of the present invention.

Physiological monitoring system 50 is connected to a mobile device 100 via cable 52 which interfaces a digital power port 54 in mobile device 100 with a digital power interface 56 in physiological monitoring system 50.

Headset 10 includes a first earbud 12 and a second earbud 14. First earbud 12a includes a sensor 60. Earbud 12 also includes an MCU 62 for controlling the operation of sensor 60 and for providing an analog signal to speaker 64. An MCU 62a can also be included within earbud 14 for converting a digital audio signal to an analog signal for speaker 64a. MCU 62 is in communication with sensor 60, speaker 64 and interface 56, respectively.

During operation, mobile device 100 transmits digital audio signals (e.g., music, phone conversation, podcasts, etc.) to headset 10. MCUs 62 and 62a convert the digital audio signals to analog audio signals and transmit the signals to speakers 64 and 64a (respectively).

Physiological data collected by sensor 60 (e.g. optical signals) is transmitted to MCU 62 which in turn transmits the signal as raw data to mobile device 100. The mobile device processes the raw signal to obtain physiological information which can be presented to the user. Alternatively, mobile device 100 collects several raw signals from several sensors (in headset 10 and on mobile device 100) and correlates between signals to derive additional information or more meaningful information from the signal of sensor 60.

Headset 10 draws power from mobile device 100 such that MCUs 62 and 62a and sensor 60 can operate while a user is listening to music. Thus, headset 10 does not require any additional power source for operation. This has the advantage of providing a low cost solution for providing physiological parameter monitoring.

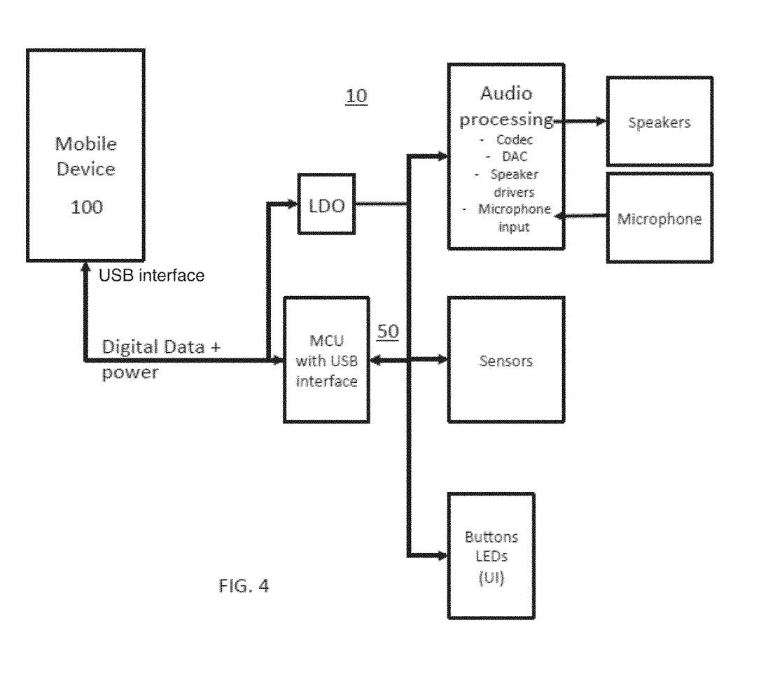

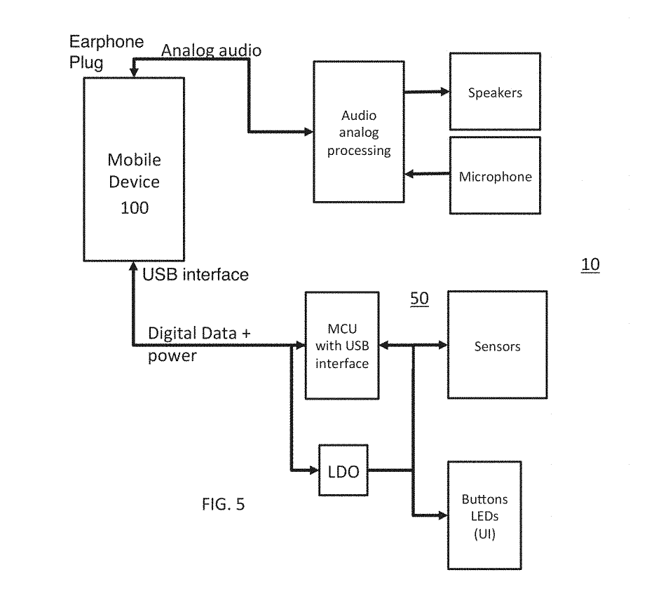

FIG. 4 is block diagram illustrating an embodiment of headset 10 which is coupled to a single port of a mobile device 100. FIG. 5 is block diagram illustrating an embodiment of headset 10 which is coupled to two ports of a mobile device 100. Both configurations also include a low dropout regulator (LDO) for regulating the voltage provided to the MCU (e.g. maintaining it between 1.8-4.7 V).

Both embodiments of headset 10 include a physiological monitoring system 50 and are connectable to a mobile phone 100 via a digital power interface. However, in the two port embodiment, headset 10 is also connected to an audio port of mobile device and thus the speakers of this headset receive an analog signal directly from mobile device 100.

In the embodiment of FIG. 4, headset 10 is connected to mobile device 100 through a single port (e.g. USB) which provides bidirectional data communication, audio signals and power. In this embodiment, headset 10 includes an audio processing component (integrated into MCU or speaker) for digital to analog conversion.

In the embodiment of FIG. 5, the audio signals are separately provided via an analog audio port of mobile device 100 thus negating the need for digital to audio conversion.

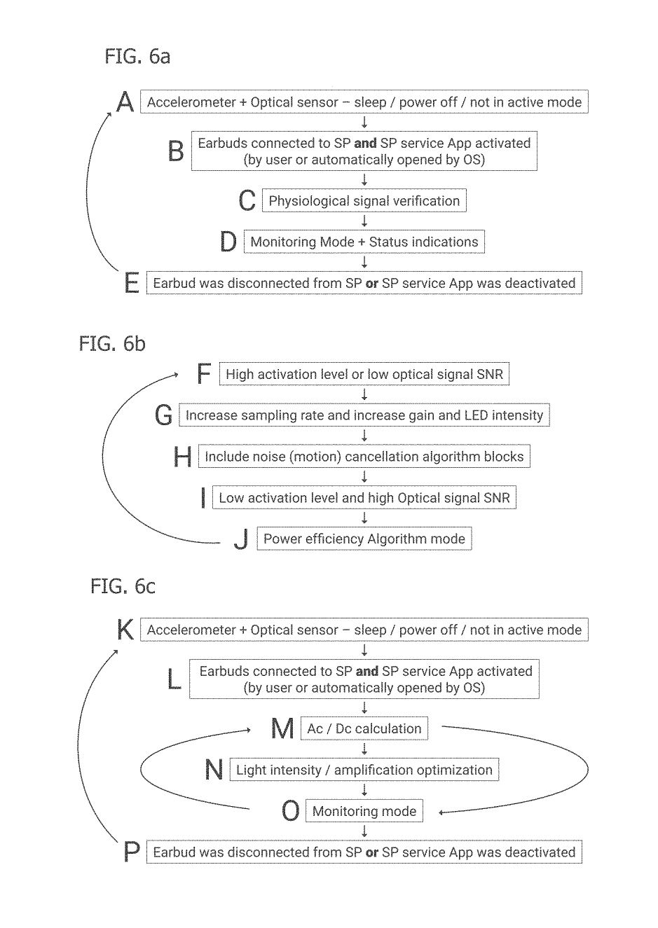

FIGS. 6a-c are flowcharts illustrating the role of the mobile device algorithm in operating and managing physiological monitoring system 50 of headset 10.

FIG. 6a describes initial calibration and data collection as performed by a service App running on the mobile device. The service App runs as a library at the application layer of the mobile device (smartphone—SP) and provides a service for other applications (e.g. provides a fitness application with a HR signal).

Step (A) is an initial state in which the sensors are not active (e.g. headset not plugged to mobile device, is in sleep mode, or mobile service App is not active). Once the headset is connected to the mobile device and the service App is running (B), the sensors activate (C) and the service App acquires data from the sensors to verify the quality of the signals (Step D). Once signal quality is verified, the service App collects sensors data and simultaneously processes the raw signal to derive HR. The service App measures and tracks the HR value while providing an indication status (i.e. measurement confidence, earbuds are out of the ear etc.—Step E). If the headset is disconnected from the mobile device, the service App deactivates the sensors or the mobile device shuts down the service App.

FIG. 6b describes the algorithm of the service App for adjusting processing resources (power consumption) and sensor function. In step F, the signal to noise ratio (SNR) of the optic signal from the PPG sensor and the motion signal from the accelerometer are analyzed, parameters such as PPG DC level stability, PPG modulation index, accelerometer magnitude STD level and more are used in this analysis. If part or a combination of those values falls below a predefined threshold, the algorithm (which runs in the mobile service App) configures the sensors for higher sampling rate and gain (amplifiers and/or light intensity) in order to improve the confidence of the HR measurement (G). When in this operational state, the HR algorithm (also part of the service App) can decide to add additional filtering blocks for noise cancelation (H) based on the SNR, such as RLS filtering at a different length and settling time. If the motion sensors (accelerometer) indicate a high motion level, a shorter RLS length will be used in order to follow/track the motion changes and with that improve motion subtraction from the PPG signal. If the activity level drops (based on accelerometer data) and the SNR level of PPG signal improves (I), the algorithm reduces the calculation level and moves to state (J)—a more efficient management of MCU resources and power consumption.

FIG. 6c describes in more detail the mechanism underlying automatic gain control (AGC) which is used to optimize the dynamic range of the sensors. When the headset is connected to mobile device (e.g. SP) and the service App is active, the sensors communicate raw signals to the HR algorithm (of the service App) through the MCU (L). Once the algorithm starts acquiring raw signals, it computes the DC level and the modulation index of the optic signal (also refer as AC/DC level) and tunes (M-N) the light intensity and amplification level of the optic sensor accordingly in order to achieve the required DC level with minimum modulation index required to compute HR in active mode. When in monitoring mode (O), the algorithm continues to check the modulation index and the DC level in order to update the light intensity and amplification level if required and to optimize the dynamic range. When the headset is disconnected from the mobile device and/or the service App is not running, the sensors are deactivated (P).

FIG. 7 illustrates a mobile device 100 (e.g. a smartphone 100) which can be used along with headset 10 of the present invention. Smartphone 100 includes a screen 110, a first input/output port 115a (e.g. USB), and a second input/output port 115b (e.g., an audio jack), internal components such as the processor and power source are not shown.

First input/output port 115a can be configured to allow (a) the battery of smartphone 100 to transmit/receive power, for example, by receiving a charge via a power charging device or transmitting a charge to a second device connected to the smartphone, and (b) the smartphone processor to transmit/receive data (e.g., for purposes of backing up the phone).

First input/output port 115a can be a micro universal serial bus (USB) port, a USB On the Go port, an Apple Lighting™ port, or any input/output port capable of bidirectional data transfer and power. The second input/output port 115b can be configured as a standard audio input/output (e.g., a 3.5 mm stereo audio jack).

Headset 10 can transmit/receive data audio signals and power via first input/output port 115a. Alternatively, headset 10 can transmit/receive data and power via first input/output port 115 and an analog audio signal via second port 115b.

Smartphone 100 is configured capable of processing raw data received from headset 10. For example, smartphone 100 can include an app that can determine heart rate and/or heart rate variability based on a raw PPG signal obtained from headset 10 and displaying the heart rate on screen 110.

The smartphone is also configured capable of setting headset functions. For example, the smartphone can:
(i) power down the MCU and sensors when the first sensor does not obtain physiological data;
(ii) power up the MCU to query one or more sensor when movement data is obtained by a movement sensor or when optical data is obtained by the PPG sensor;
(iii) set a sampling rate for the sensors based on movement data or a signal to noise ratio of movement and PPG data as processed by the mobile device;
(iv) modify an amplification and sampling rate of the PPG sensor according to a skin tone of the user;

As used herein the term “about” refers to ±10%.

Additional objects, advantages, and novel features of the present invention will become apparent to one ordinarily skilled in the art upon examination of the following examples, which are not intended to be limiting.

### EXAMPLES

Reference is now made to the following examples, which together with the above descriptions, illustrate the invention in a non limiting fashion.

Twelve healthy subjects 19 to 45 years old performed forced breathing, stand-up and 70° tilt-up tests (each for 3 times in different patterns). Ear PPG (400 Hz) and Acceleration (400 Hz) as well as smartphone acceleration (400 Hz) from the arm were measured continuously as well as HR from a chest strap (as a control). Beat to Beat interval, Peak to Peak and DC level were computed from the PPG signal and the dynamics of the stand-up test were calculated from the 2 accelerometers (earbud accelerometer and smartphone accelerometer) in order to identify the effect of a standup pattern on HR.

Results

A positive association was found between PPG characteristics and the acceleration pattern of the standup. Moreover, there was a significant variance in the PPG characteristics between the 3 standups indicating acceleration had a strong effect on cardiovascular response. The HR results obtained from a chest strap did not demonstrate any significant variance.

Conclusions

The ability to correlate raw PPG signals with raw accelerometer signals during a standup test can enhance analysis of cardiovascular response to physical stress and provide valuable insight into a patient's cardiovascular health.

Such insights cannot be gained from correlating HR from a chest strap (ECG) with accelerometer signals since unlike the PPG signal, the ECG signal simply reflects HR and does not include additional information indicating, for example, vasoconstriction or vasodilation which can be associated with the acceleration pattern.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A headset comprising:
(a) at least one earbud including an audio speaker and:
(i) a first sensor for obtaining physiological data from said subject when positioned against a tissue region of an ear;
(ii) a second sensor for obtaining movement data from said subject;
(b) a microcontroller being for:
(i) obtaining said physiological data and said movement data from said first sensor and said second sensor when said first sensor is positioned against said tissue region of said ear;
(ii) converting a digital input signal to an analog output signal for driving said audio speaker; and
(d) a digital interface for connecting said at least one earbud to a mobile device via a wire, said interface being configured for:
(i) enabling said mobile device to power said microcontroller and said first sensor and said second sensor;
(ii) communicating a digital audio signal from said mobile device to said microcontroller; and
(iii) communicating said physiological data and said movement data obtained by said microcontroller to said mobile device via said wire as a raw digital signal to thereby enable a processor of said mobile device to extract physiological and movement information from said data.

2. The headset of claim 1, wherein said first sensor and said second sensor are positioned in or on a region of said at least one earbud subjected to a uniform movement pattern.

3. The headset of claim 1, wherein said second sensor obtains movement data from at least two movement axis.

4. The headset of claim 1, wherein said first sensor is an optical sensor.

5. The headset of claim 4, wherein optical sensor includes at least two light emitting diodes (LEDs).

6. The headset of claim 5, wherein each of said at least two LEDs is capable of emitting light at a different wavelength.

7. The headset of claim 5, wherein each of said at least two LEDs is positioned at a different distance from a photodetector.

8. The headset of claim 1, wherein positioning said first sensor in an ear of said subject generates a signal for executing an action in said mobile device.

9. The headset of claim 1, wherein said physiological data includes heart rate data.

10. The headset of claim 1, wherein said physiological data includes SpO₂ data, blood pressure data, heart rate data, body temperature data and/or bioimpedance data.

11. The headset of claim 1, wherein said first sensor is selected from the group consisting of a temperature sensor, a galvanic skin resistance sensor, a blood pressure sensor, an ECG sensor, an EOG sensor, an EEG sensor and a bioimpedance sensor.

12. The headset of claim 1, wherein said second sensor is selected from the group consisting of an accelerometer, a gyroscope, magnometer.

13. The headset of claim 1, wherein said microcontroller is further configured for converting signals from said first sensor and said second sensor to a digital format of said digital interface.

14. A system comprising:
(a) a headset having at least one earbud including:
(i) an audio speaker;
(ii) a sensor for obtaining physiological data from a subject when positioned against a tissue region of an ear;
(iii) a microcontroller for obtaining said physiological data from said first sensor and said second sensor when said sensor is positioned against said tissue region of said ear and relaying said physiological data over a wired connection as a raw signal; and
(b) a mobile device having an on-board sensor and being wired to said headset via a digital interface, wherein a processor of said mobile device processes said raw signal along with a second raw signal from said on-board sensor to thereby derive information resulting from a correlation between said raw signal and said second raw signal.

15. The system of claim 14, wherein said mobile device powers down said microcontroller and said first sensor when physiological data is not obtained.

16. The system of claim 14, wherein said headset further comprises a movement sensor.

17. The system of claim 16, wherein said mobile device powers up said microcontroller to query said first sensor when movement data is obtained by said movement sensor and/or when said physiological data is obtained by said first sensor.

18. The system of claim 17, wherein said mobile device sets a sampling rate for said physiological data and said movement data.

19. The system of claim 18, wherein said sampling rate is dependent on movement data or a signal to noise ratio of said physiological data and/or said movement data.

20. The system of claim 14, wherein said physiological sensor is an optical sensor.

21. The system of claim 20, wherein a light intensity, amplification and sampling rate of said optical sensor is modifiable by said mobile device according to a skin tone of said subject.
